(19) United States
(12) Patent Application Publication
(10) Pub. No.: US 2021/0007861 A1
(43) Pub. Date: Jan. 14, 2021

(54) INSERTION ANGLE-ADJUSTABLE IMPLANT CAGE FOR OBLIQUE LUMBAR INTERBODY FUSION SURGERY

(71) Applicant: THE CATHOLIC UNIVERSITY OF KOREA INDUSTRY-ACADEMIC COOPERATION FOUNDATION, Seoul (KR)

(72) Inventor: Jin Sung KIM, Yongin-Si Gyeonggi-do (KR)

(73) Assignee: THE CATHOLIC UNIVERSITY OF KOREA INDUSTRY-ACADEMIC COOPERATION FOUNDATION, Seoul (KR)

(21) Appl. No.: 17/042,282

(22) PCT Filed: Dec. 12, 2018
(86) PCT No.: PCT/KR2018/015749

§ 371 Date: Sep. 28, 2020

(30) Foreign Application Priority Data
Apr. 30, 2018 (KR) 10-2018-0049801

(51) Int. Cl. A61F 2/44

(52) U.S. Cl. CPC A61F 2/447; A61F 2002/30578

(57) ABSTRACT

The present invention provides a vertebral implant cage for oblique lumbar interbody fusion surgery, comprising: a first member (110) to be inserted between vertebral bodies; a second member (120) coupled to a front surface of the first member; a screw hole (125) formed through the second member; a screw (130) fastened to the screw hole; and a vertical coupling hole (115) vertically formed through the first member so as to couple the second member thereto, wherein the second member can slide a predetermined distance along the front circumference of the first member while being coupled to the vertical coupling hole.

### TECHNICAL FIELD

The present invention relates to an implant cage for a vertebral body insertion, to an implant cage to be inserted into a position created by the removal of a disc that formed a spine of a human body, to serve as the disc, and to an implant cage for use in an oblique lumbar interbody fusion (OLIF) surgery in which an intervertebral fusion cage is inserted by way of the oblique lumbar.

### BACKGROUND ART

When a disc that forms a spine of a human body is unable to perform its original function due to degeneration, diseases from the degeneration, aging, accident, and the like, the disc is removed and replaced with a substitute that is inserted into the position created by the removal of the disc to perform the function of the disc. The disc between the vertebrae and the bone serves as a joint and plays a very important role in minimizing the impact on the spine, with the position and shape of the nucleus pulposus contained in the disc changing according to a movement of the spine. Most of the nucleus pulposus is made up of water, and the amount of water gradually decreases as aging continues and the disc can even lose its buffering function. For this reason, the fiber under excessive pressure results in back pain, and when this continues, a severe stretching of the fiber or rupture may occur, causing the nerve root located at the back to be pressed to cause pain in the pelvis, legs, and the like.

As a method of treating a disease caused by the disc, there is a method of removing the damaged intervertebral disc and replacing a space between two adjacent vertebraes with a cage. Examples of such a surgical method include an anterior lumbar interbody fusion (ALIF) that opens the abdomen and inserts a cage in a front side of the spine, a lateral lumbar interbody fusion (LLIF) that inserts a cage through the flank, a transforaminal lumbar interbody fusion (TLIF) that inserts a cage diagonally at a point 30 to 40 mm away from the center of the back to the side, a posterior lumbar interbody fusion (PLIF) that inserts a cage from the back, and the like.

Korean Registered Patent No. 10-1040515 discusses a related art which is illustrated in FIG. 1. The configuration of the related art mainly includes a first body 110 in a “C” shape including a cage 100 formed with a hollow portion into which the main chip is to be inserted, a second body 120 coupled to the first body 110, and a screw 500 coupled to the vertebral body.

However, in the related art, since the relative position of the second body to the first body forming the C shape is fixed, there is a problem that it is difficult to move the position of the second body with the first body being maintained in the fixed state. This issue is particularly challenging in an oblique lumbar interbody fusion surgery (hereinafter referred to as ‘OLIF surgery’), because, while the ideal oblique angle varies patient by patient, it is very important for the OLIF surgery using the oblique trajectory to find the optimal oblique trajectory and fix the same at the actual surgical field, thus requiring that a member (the second body described above) to which the screw is fixed be allowed to have a certain degree of relative movement in order to find the optimal oblique trajectory.

However, the related arts had a problem that such a relative movement was impossible.

### DISCLOSURE

### Technical Problem

An object of the present invention is to provide a means for finding an optimal implant cage mounting angle that is most ideal for each of the patients and fixing a cage accordingly, in the process of inserting a vertebral implant cage in an oblique lumbar interbody fusion.

### Technical Solution

The present invention provides a vertebral implant cage for oblique lumbar interbody fusion surgery, which may include: a first member 110 to be inserted between vertebral bodies; a second member 120 coupled to a front surface of the first member; a screw hole 125 formed through the second member; a screw 130 fastened to the screw hole; and a vertical coupling hole 115 vertically formed through the first member so as to couple the second member thereto, in which the second member can slide a predetermined distance along the front circumference of the first member while being coupled to the vertical coupling hole.

Further provided may include a coupling protrusion 122 protruding backward from a back surface of the second member; and a vertical bar 140 coupled to the coupling protrusion of the second member and mounted to the vertical coupling hole 115.

Further provided may include a horizontal coupling hole 118 formed in the transversal direction in the front surface of the first member and communicating with the vertical coupling hole.

The second member may include a second-first member 120a with a coupling protrusion formed thereon; and a second-second member 120b and a second-third member 120c provided above and below the second-first member to receive upper and lower ends of the second-first member inserted therein, in which the second-first member 120a includes a central portion 120a-1, an upper portion 120a-2 and a lower portion 120a-3 extended upward and downward from the central portion, and the widths of the upper portion 120a-2 and the lower portion 120a-3 are smaller than the width of the central portion 120a-1.

In addition, the present invention provides a vertebral implant cage for oblique lumbar interbody fusion, which may include: a first member 110 to be inserted between vertebral bodies; a second member 220 coupled to a front surface of the first member; a coupling space 115′ formed in the first member; and a second coupling protrusion including a horizontal member 222 and a vertical member 223 on a back surface of the second member, and coupled to the coupling space, in which the second member is slidable a predetermined distance along a front circumference of the first member, with a second coupling protrusion being coupled to the coupling space.

Further provided may include a guide groove 115a formed in a transversal direction on an inner surface of the first member coupling space 115′; and a guide protrusion 224 protruding from a vertical member 223 of the second member to be inserted into the guide groove 115a.

The guide groove may be formed on both front and back surfaces of the inner surface of the first member coupling space 115′, and the guide protrusion may be formed in both forward and backward directions of the vertical member 223 of the second member.

### Advantageous Effects

With the configuration described above, the present invention provides an advantageous effect that it is possible to find an optimal implant cage mounting angle that is most ideal for each of the patients and fix a cage accordingly, in the process of inserting the vertebral implant cage in an oblique lumbar interbody fusion.

### BRIEF DESCRIPTION OF THE DRAWINGS

FIG. 1 is a perspective view of an intervertebral fusion cage according to a related art.

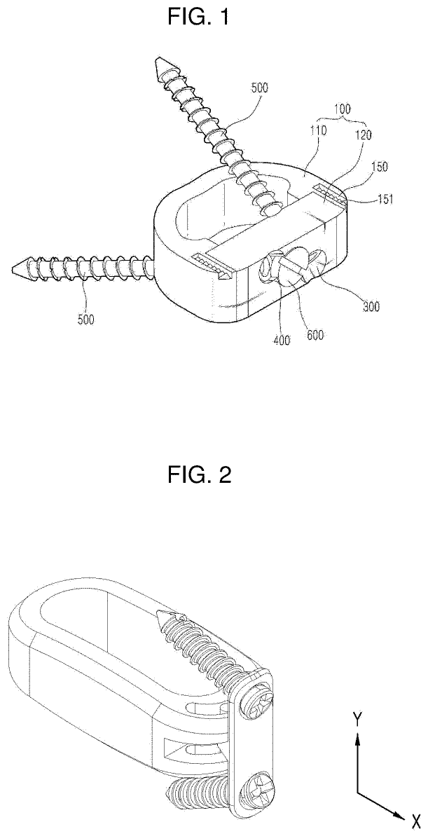

FIG. 2 is a perspective view of a vertebral implant cage for oblique lumbar interbody fusion (OLIF) surgery according to a first embodiment of the present invention.

FIG. 3 is a perspective view of the vertebral implant cage in disassembled state according to the first embodiment of the present invention.

FIG. 4 is a perspective view of some components of the vertebral implant cage according to the first embodiment of the present invention.

FIG. 5 shows the vertebral implant cage according to the first embodiment of the present invention in sliding motion.

FIG. 6 shows the vertebral implant cage according to the first embodiment of the present invention inserted between vertebral bodies.

FIGS. 7 and 8 show some modifications of the vertebral implant cage according to the first embodiment of the present invention.

FIG. 9 shows the vertebral implant cage of FIG. 7 inserted between vertebral bodies.

FIG. 10 shows a vertebral implant cage according to a second embodiment of the present invention.

FIG. 11 shows some components of the vertebral implant cage according to the second embodiment of the present invention.

FIGS. 12 and 13 show some modifications of the vertebral implant cage according to the second embodiment of the present invention.

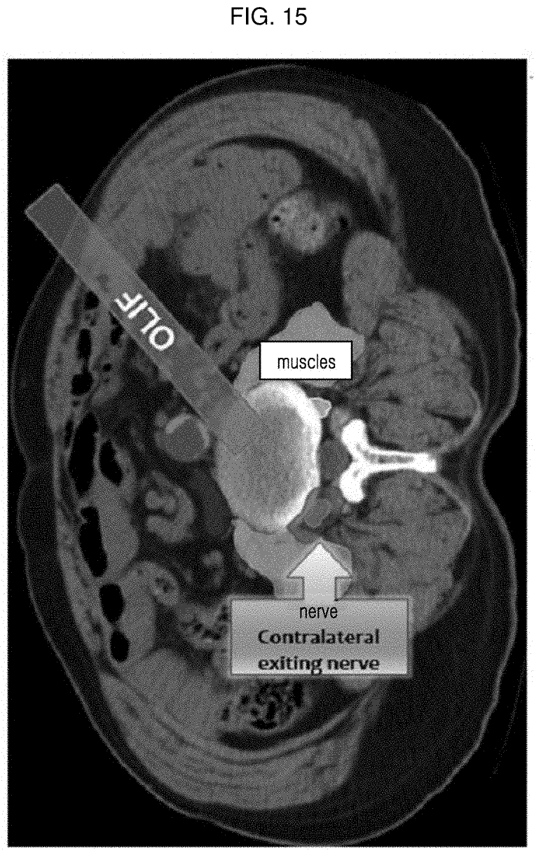

FIGS. 14 to 16 are views illustrating a path for surgery to explain features of the OLIF surgery to which the present invention pertains.

FIG. 17 shows mounting of the implant cage of the OLIF surgery to which the present invention pertains, in contrast to an ideal direction.

### BEST MODE

The objects, specific advantages and novel features of the present invention will be more apparent from the following detailed description and preferred embodiments associated with the accompanying drawings. In addition, the terms used herein are those defined in consideration of functions in the present invention, which may vary according to the intent or practice of the user/operator. Therefore, the definitions of these terms should be made based on the contents throughout the description.

### First Embodiment

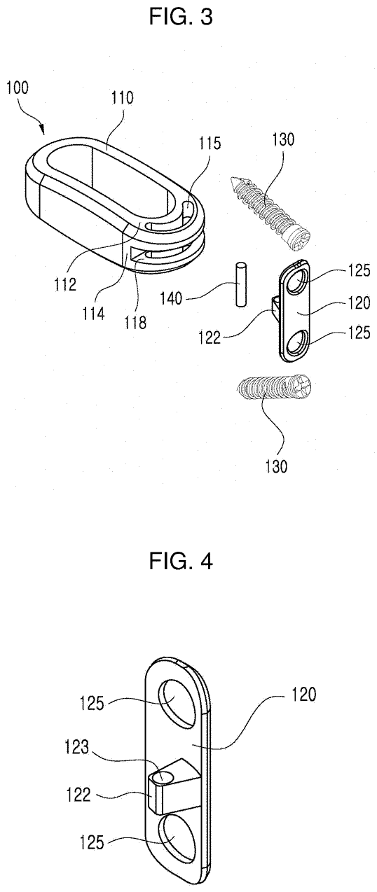

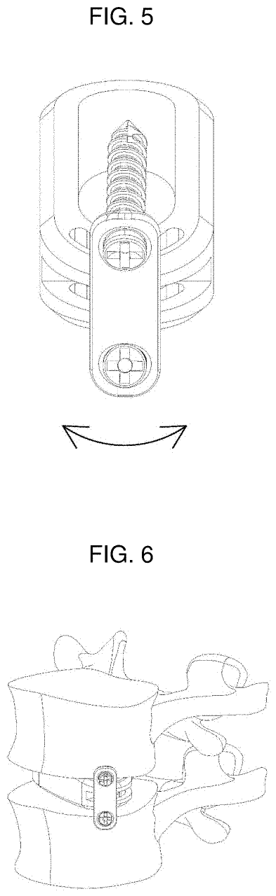

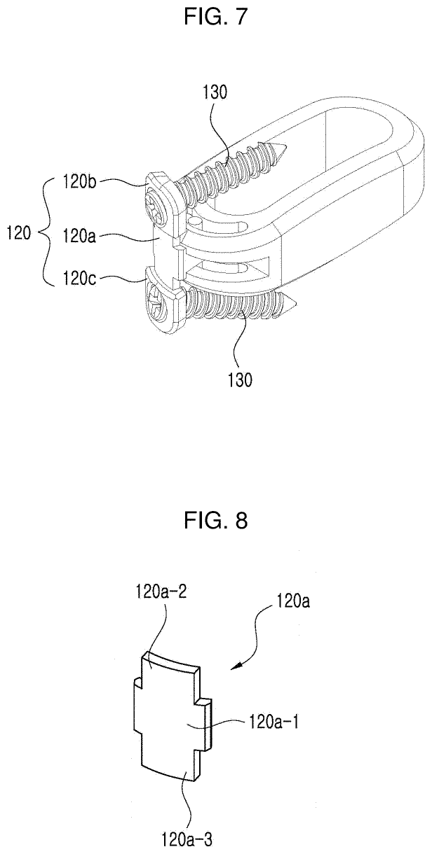

FIG. 2 is a perspective view of a vertebral implant cage (hereinafter referred to as an ‘implant cage’) for oblique lumbar interbody fusion (OLIF) surgery according to a first embodiment of the present invention, FIG. 3 is a perspective view of the implant cage in disassembled state according to the first embodiment of the present invention, and FIG. 4 is a perspective view of some components of the implant cage according to the first embodiment of the present invention

The implant cage 100 according to FIGS. 2 to 4 includes a first member 110 which is a part inserted between the vertebral bodies, a second member 120 coupled to a front surface of the first member 110 and fixed to the vertebral body with a screw 130. Among the expressions used herein to indicate the directions, when ‘forward and backward’ is used, for convenience, it is assumed that the forward direction is a direction (x direction in FIG. 2) from the first member of the implant cage toward the second member, and the backward direction is an opposite direction thereto. It is further assumed that, in FIG. 2, an upward direction refers to the upper side (y-direction of the drawing), and a downward direction refers to an opposite direction thereto. A screw hole 125 for mounting the screw 130 for fixing the vertebral body is formed on upper and lower surfaces of the second member 120.

The first member 110 is shaped such that it includes an inner space in a circular or elliptical shape as a whole when viewed from the top, and has a vertical coupling hole 115 formed in a front thickness portion in a up-and-down direction to be coupled with the second member 120. The first member has an elliptical shape when viewed from the top, so that its outer border forms a curved portion. That is, the vertical coupling hole 115 is a hole formed from the upper surface 112 to the lower surface of the first member in the vertical direction while forming a curved portion along the outer curved portion of the first member.

In addition, the first member 110 further includes a horizontal coupling hole 118 formed in a transversal direction from the front surface 114 of the first member toward the back side, and communicating with the vertical coupling hole 115. The first member and the second member are coupled by a vertical bar 140. The second member 120 includes a coupling protrusion 122 protruding backward from the back surface, and the vertical bar 140 is coupled with the coupling protrusion 122. The coupling protrusion 122 of the second member is a structure that is formed with a coupling hole 123 to receive the vertical bar to be inserted therein. The coupling protrusion protrudes from the back surface of the second member and the coupling protrusion 122 is structured such that it is fitted in the horizontal coupling hole 118 of the first member.

That is, when the first member and the second member are coupled to each other, with the coupling protrusion 122 being fitted in the horizontal coupling hole 118, the vertical bar 140 is vertically mounted to the vertical coupling hole 115, and at the same time, the vertical bar 140 is inserted into and fixed in the coupling protrusion 122.

As described above, the main feature of the present invention is that the first member 110 and the second member 120 are coupled to each other via the vertical bar, such that the second member is slidably moved a predetermined distance along a front circumference of the first member with the first and second members being coupled to each other.

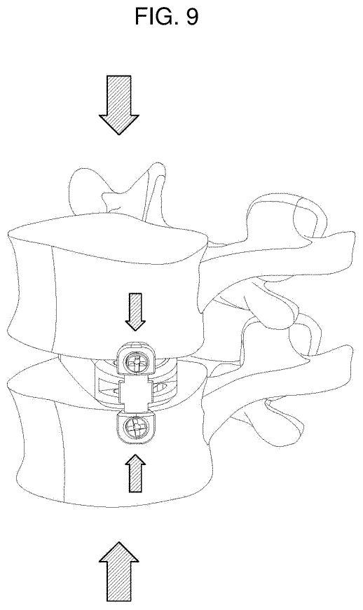

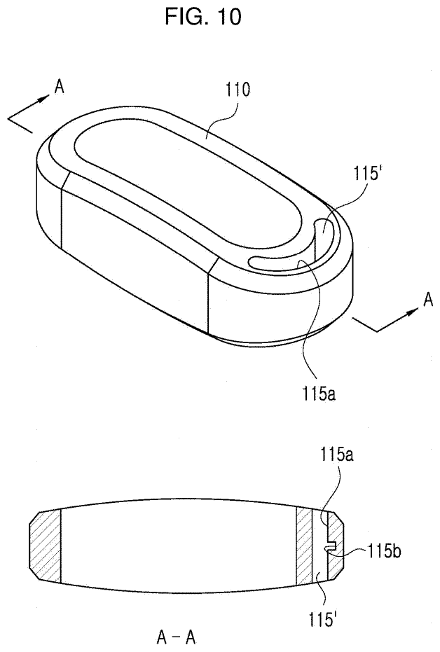

FIG. 5 shows the vertebral implant cage according to the first embodiment of the present invention in sliding motion, FIG. 6 shows the vertebral implant cage according to the first embodiment of the present invention inserted between vertebral bodies. There the reason for the second member being slidable a predetermined distance while the first and second members are coupled to each other, and this will be described below in relation to the characteristics of the OLIF surgery.

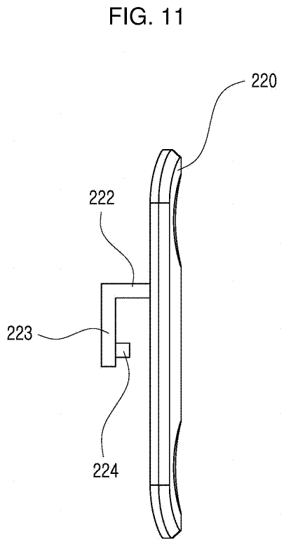

FIGS. 7 and 8 show some modifications of the vertebral implant cage according to the first embodiment of the present invention. Referring to FIGS. 7 and 8, since the first member 110 among the components of the implant cage has the same structure as that described above, a description thereof will be omitted. Referring to FIG. 7, the difference is that the second member is changeable in its length in the up-and-down direction (vertical direction) while it is coupled to the vertebral body with the screw, and such change in the length in the up-and-down direction is allowed to enable the implant cage to cope with the subsidence of the vertebral body.

The coupling protrusion 122 described above is likewise formed on the second member 120 shown in FIG. 7. However, the second member of the present embodiment is characterized in that it includes a second-first member 120a in the middle, and a second-second member 120b and a second-third member 120c coupled above and below, and these three members are each formed as separate members and coupled to each other, and when in the coupled state, the second-second member 120b and the second-third member 120c are relatively movable in the up-and-down direction, such that the overall size of the second member in the up-and-down direction is variable. The coupling protrusion 122 is formed behind the second-first member 120a that is positioned in the middle, and the screw hole to receive the screw to be mounted is formed on the second-second member 120b and the second-third member 120c.

Specifically, the second-first member 120a includes a central portion 120a-1, an upper portion 120a-2 and a lower portion 120a-3 extended upward and downward from the central portion, in which the widths of the upper portion 120a-2 and the lower portion 120a-3 are smaller than the width of the central portion 120a-1. Due to such a difference in width, the limits for the second-second member 120b and the second-third member 120c to move in the up-and-down direction are determined. The ends of the upper portion 120a-2 and the lower portion 120a-3 are respectively inserted into the second-second member 120b and the second-third member 120c, respectively, and for this, grooves (not shown) for insertion and mounting are formed on each of the lower end of the second-second member 120b and the upper end of the second-third member 120c.

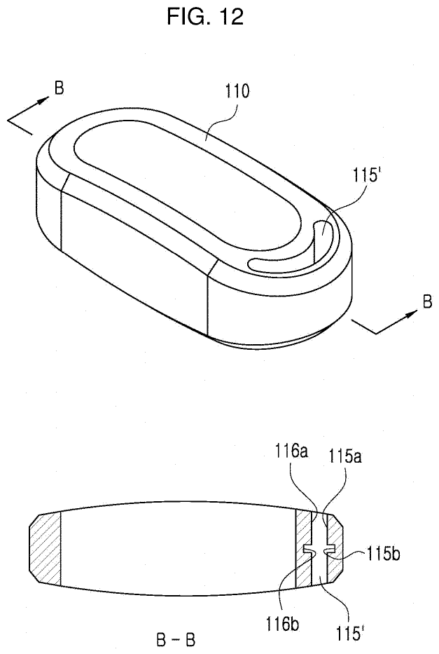

FIG. 9 shows the vertebral implant cage of FIG. 7 inserted between the vertebral bodies and moved in the up-and-down direction.

### Second Embodiment

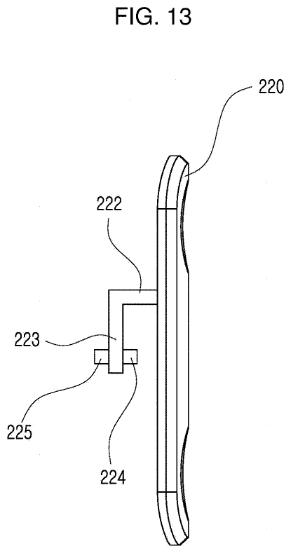

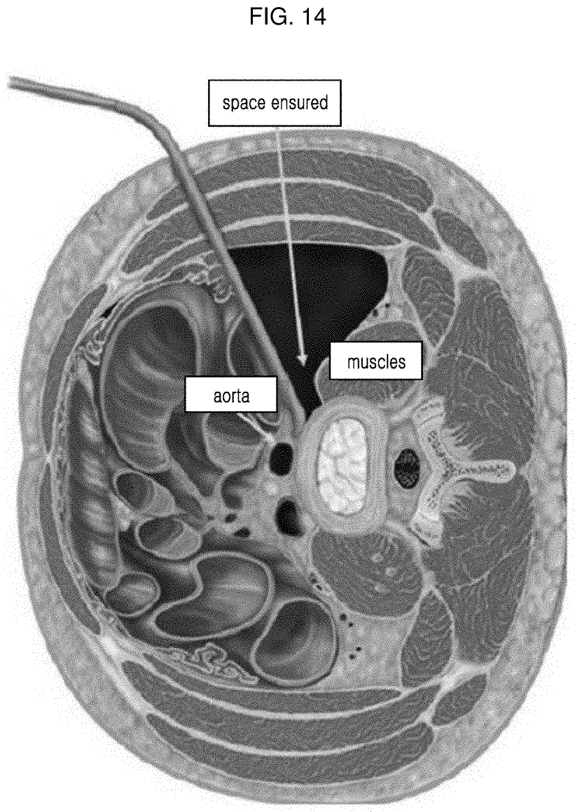

FIG. 10 shows a vertebral implant cage according to a second embodiment of the present invention, and FIG. 11 shows some components of the vertebral implant cage according to the second embodiment of the present invention.

The vertebral implant cage according to the second embodiment of the present invention includes a first member 110 to be inserted between vertebral bodies, a second member 220 coupled to a front surface of the first member, a screw for fixing the second member to the vertebral body, and the like.

The first member 110 is shaped such that it includes an inner space in a circular or elliptical shape as a whole when viewed from the top, and has a coupling space 115′ formed in a front thickness portion in the up-and-down direction to be coupled with the second member 220. The first member has an elliptical shape when viewed from the top, such that an outer border forms a curved portion, and the coupling space 115′ also forms a curved portion along the outer curved portion of the first member. The coupling space 115′ may be a hole that is extended completely through, from the upper surface 112 to the lower surface of the first member in the vertical direction, and may also be a groove formed only a predetermined depth from the upper surface.

Second coupling protrusions 222 and 223, including the horizontal member 222 and the vertical member 223, are formed on the back surface of the second member 220 and coupled to the coupling space 115′. The second member is coupled to the first member as the second coupling protrusion is fitted into the coupling space. Then, with the second coupling protrusion being coupled to the coupling space, the second member is slidable a predetermined distance along the front circumference of the first member. The need for the sliding motion will be described below.

In this embodiment, a structure for guiding a sliding motion is further included, which includes a guide groove 115b formed in the transversal direction on a front surface 115a of the inner surface of the first member coupling space 115′. In addition, the second member 220 includes a guide protrusion 224 protruding forward from the lower end of the vertical member 223 to be inserted into the guide groove 115b. The guide protrusion 224 is fitted in the guide groove 115b to allow a sliding motion in the transversal direction.

The guide groove may be formed on either or both of the front or back side of the inner surface of the coupling space, and the guide protrusion may also be formed on either or both of the front or back side of the vertical member 223. The drawing shows the guide grooves 115b and 116b being formed on both the front surface 115a and the back surface 116a of the coupling space 115′, and the guide protrusions 224 and 225 protruding forward and backward from the lower end of the vertical member 223 of the second member 220 to be inserted into the guide groove.

In this embodiment, since the guide protrusion is inserted into the guide groove to guide the sliding motion, the sliding movement may be performed along a predetermined transverse path, and the degree of smoothness of the sliding motion may be defined by appropriately selecting the sizes of the guide groove and the guide protrusion, and also, the section (width) in which the sliding motion is performed may be defined. That is, by defining the length of the transversal direction in which the guide groove is formed and forming a guide groove to correspond to this size, the section for sliding can be determined.

[Reason for Requiring Sliding Motion in OLIF Surgery]

The main feature of the present invention is that the second member 120 is capable of relative sliding, with the first member 110 of the implant cage being mounted between the vertebral bodies. Hereinafter, the reason for needing such a sliding motion will be described in relation to the characteristics of the OLIF surgery.

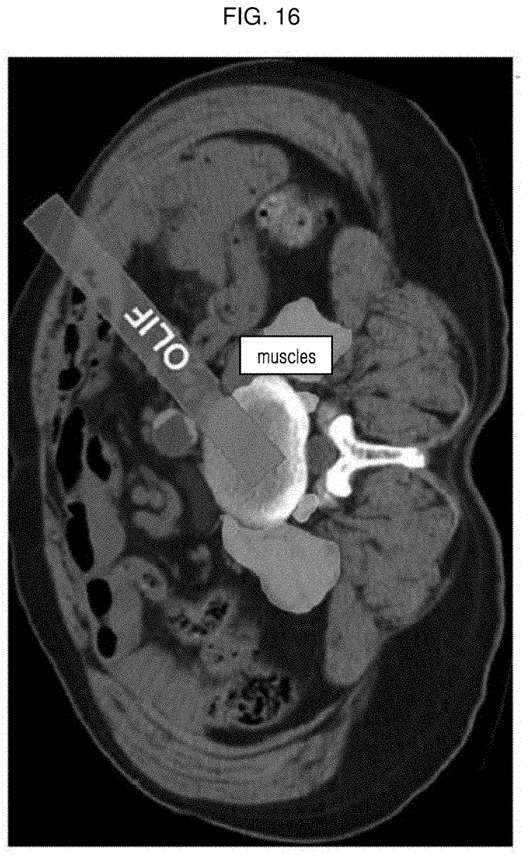

FIGS. 14 to 17 show a path for performing an OLIF surgical procedure to which the present invention pertains, and shows an implant cage being mounted in the surgical procedure.

First, FIG. 14 shows a path for performing OLIF surgery, and a surgical tool is used to ensure a space between the aorta and the psoas muscle, and an implant cage is mounted through the ensured space. However, directly entering the path in the ensured space may pose a risk of possible damages to the contralateral exiting nerve (see FIG. 15). Accordingly, in OLIF surgery, the OLIF surgery is performed after retraction of the psoas muscle (see FIG. 16). However, excessive retraction of the psoas muscle increases the likelihood of associated complications, and therefore, the psoas muscle can not be retracted excessively.

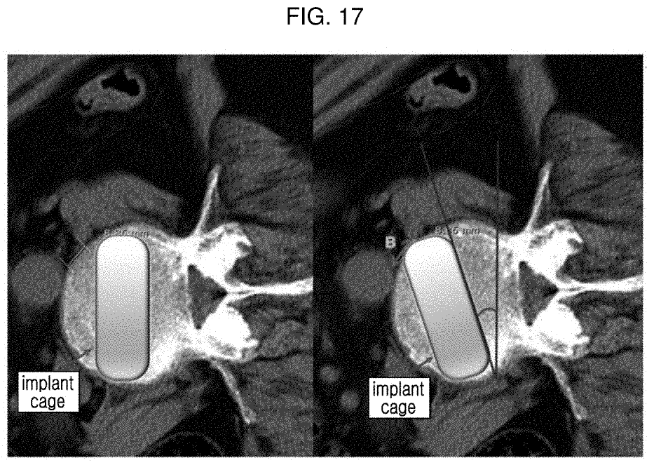

FIG. 17 shows the position of the ideal implant cage (left-hand side view) in contrast to the position of the actually mounted implant cage (right-hand side view). If the psoas muscle can be excessively retracted, it would be possible to mount the implant cage in the same direction as the left-hand side view in FIG. 17, but due to the limited amount of retraction of the psoas muscle, the implant cage is inserted at a slightly oblique angle as shown in the right-hand side view.

In the course of surgery, the angle at which the implant cage is mounted (FIG. 17A) is determined by considering various conditions such as length (red arrow B) of the OLIF corridor, a degree of development (size) of the psoas muscle of a patient, a relationship between shapes of the vertebral body and the psoas muscle, and the like, and all of these factors are different patient by patient in surgery and need to be confirmed during the surgery. Therefore, after inserting the first member 110 of the implant cage and positioning the second member in an optimal position, it is necessary to perform the sliding motion of the second member to fix the screw to the vertebral body.

What is claimed is:

1. An inserting angle-adjustable implant cage for oblique lumbar interbody fusion surgery, comprising:
a first member (110) inserted between vertebral bodies;
a second member (120) coupled to a front surface of the first member;
a screw hole (125) formed through the second member;
a screw (130) fastened to the screw hole; and
a vertical coupling hole (115) vertically formed through the first member so as to couple the second member thereto,
wherein the second member is slidable a predetermined distance along a front circumference of the first member, while it is coupled to the vertical coupling hole.

2. The inserting angle-adjustable implant cage according to claim 1, further comprising:
a coupling protrusion (122) protruding backward from a back surface of the second member; and
a vertical bar (140) coupled to the coupling protrusion of the second member and mounted to the vertical coupling hole (115).

3. The inserting angle-adjustable implant cage according to claim 2, further comprising a horizontal coupling hole (118) formed in a transversal direction in the front surface of the first member and communicating with the vertical coupling hole.

4. The inserting angle-adjustable implant cage according to claim 3, wherein the coupling protrusion (122) of the second member includes a coupling hole (123) formed in an up-and-down direction to receive the vertical bar inserted thereto.

5. The inserting angle-adjustable implant cage according to claim 1, wherein the second member includes:
a second-first member (120a) with a coupling protrusion formed thereon; and
a second-second member (120b) and a second-third member (120c) provided above and below the second-first member to receive upper and lower ends of the second-first member inserted therein.

6. The inserting angle-adjustable implant cage according to claim 5, wherein the second-first member (120a) includes a central portion (120a-1), an upper portion (120a-2) and a lower portion (120a-3) extended upward and downward from the central portion, and
widths of the upper portion (120a-2) and the lower portion (120a-3) are smaller than a width of the central portion (120a-1).

7. An inserting angle-adjustable implant cage for oblique lumbar interbody fusion surgery, comprising:
a first member (110) inserted between vertebral bodies;
a second member (220) coupled to a front surface of the first member;
a coupling space (115′) formed in the first member; and
a second coupling protrusion including a horizontal member (222) and a vertical member (223) on a back surface of the second member, and coupled to the coupling space,
wherein the second member is slidable a predetermined distance along a front circumference of the first member, with a second coupling protrusion being coupled to the coupling space.

8. The inserting angle-adjustable implant cage according to claim 7, further comprising:
a guide groove (115a) formed in a transversal direction on an inner surface of the first member coupling space (115′); and
a guide protrusion (224) protruding from a vertical member (223) of the second member to be inserted into the guide groove (115a).

9. The inserting angle-adjustable implant cage according to claim 8, wherein the guide groove is formed on both front and back surfaces of an inner surface of the first member coupling space (115′), and
the guide protrusion is formed in both forward and backward directions of the vertical member (223) of the second member.

10. The inserting angle-adjustable implant cage according to claim 7, further comprising:
a screw hole formed through the second member; and
a screw fastened to the screw hole.

11. The inserting angle-adjustable implant cage according to claim 7, wherein the second member (220) includes:
a second-first member with the second coupling protrusion formed thereon; and
a second-second member and a second-third member provided above and below the second-first member to receive upper and lower ends of the second-first member inserted therein.

12. The inserting angle-adjustable implant cage according to claim 11, wherein the second-first member includes a central portion, and an upper portion and a lower portion extended upward and downward from the central portion, and
widths of the upper and lower portions are smaller than a width of the central portion.
